(19) United States
(12) Patent Application Publication
(10) Pub. No.: US 2009/0005750 A1
(43) Pub. Date: Jan. 1, 2009

(54) Discharge absorbing apparatus

(76) Inventor: Beverley West, Lake Worth, FL (US)

Correspondence Address: JAMES RAY & ASSOCIATES, 2640 PITCAIRN ROAD, MONROEVILLE, PA, 15146, US

(21) Appl. No.: 12/157,520

(22) Filed: Jun. 11, 2008

Related U.S. Application Data
(60) Provisional application No. 60/946,544, filed on Jun. 27, 2007.

(51) Int. Cl. A61F 13/62

(52) U.S. Cl. 604/385.03

(57) ABSTRACT

A discharge absorbing apparatus is provided. Such discharge absorbing apparatus includes an absorbent member manufactured from a first predetermined material and having a first predetermined size and a first predetermined shape. A securing mechanism is operably connected to such absorbent member at a predetermined location thereon for releasably securing such absorbent member to at least one of a person and a tube type member.

### CROSS REFERENCE TO RELATED APPLICATION

This patent application is related to and claims priority from U.S. Provisional Patent Application Ser. No. 60/946,544 filed Jun. 27, 2007.

### FIELD OF THE INVENTION

The present invention relates, in general, to feminine products for collecting and absorbing vaginal discharge, more particularly, this invention relates to a discharge absorbing apparatus for attachment to a catheter or similar device.

### BACKGROUND OF THE INVENTION

Prior to the conception and development of the present invention, feminine napkins, as are generally well known in the prior art, are often used by menstruating women and women who have just given birth, however, such products are not practical for use by women who are utilizing a catheter. Therefore there is a need for a discharge absorbing apparatus which can be utilized by a woman who is simultaneously undergoing catheterization.

Specifically of interest to the present invention are the following: Gross, U.S. Pat. No. 4,784,647, discloses a metal pad for use with a catheter applied to a urinary tract for drainage thereof. The pad comprises a cylindrically shaped block of foam material coated on one longitudinal end with covering of silicone, latex, or polyurethane. The pad and coating have a longitudinal bore therethrough, which has a slit radially directed therefrom, to facilitate mating of the pad with a catheter shaft.

Sessions, et al, U.S. Pat. No. 4,069,826, discloses a surgical tube-adapter clamp for effecting a tube connection to a blood vessel, in which a tubular member is constructed for insertion of its free end into a blood vessel and the opposite end adapted to be operatively connected to the cooperable end of a tube, in which a pair of relatively deformable members, encircle the tubular member with one of such members being adapted to be disposed adjacent the exterior wall of the blood vessel and the other member having a portion disposed within the blood vessel and adapted to be deformed to increase the effective diameter thereof within the blood vessel and thereby clamp the adjacent wall of such vessel between the two deformable members to secure the clamp to the vessel.

Marshall, et al, U.S. Pat. No. 4,516,968, discloses a catheter shield comprising a dome surrounded by a peripheral, skin engaging flange. The dome defines a cavity, has a hole for the catheter, and has a radial slit extending to the edge of the shield to aid in placement of the shield around the catheter.

Layman-Spillar, et al, U.S. Pat. No. 5,554,106, discloses a dressing which is preferably constructed of a layer of hydrocolloid material having a downwardly extending lip member thereon that is sized to be received adjacent to a percutaneous device which is at least partially implanted into a patient such that the dressing having a lip member thereon forms a physical barrier to decrease the incidence of catheter tract infections in a patient and the lip member provides a downward extension into the wound to help remove detritus from the catheter tract and wherein the dressing further includes a mesh layer adjacent to the skin of the patient to minimize the amount of hydrocolloid material remaining on the skin of the patient after use.

Gisselberg, et al, U.S. Pat. No. 5,569,207, discloses a dressing which is preferably constructed of a layer of hydrocolloid material having a downwardly extending lip member thereon that is sized to be received adjacent to a percutaneous device which is at least partially implanted into a patient such that the dressing having a lip member thereon forms a physical barrier to decrease the incidence of catheter tract infections in a patient and the lip member provides a downward extension into the wound to help remove detritus from the catheter tract and wherein the dressing further includes a mesh layer adjacent to the skin of the patient to minimize the amount of hydrocolloid material remaining on the skin of the patient after use.

### SUMMARY OF THE INVENTION

The present invention provides a discharge absorbing apparatus. Such discharge absorbing apparatus includes an absorbent member manufactured from a first predetermined material and having a first predetermined size and a first predetermined shape. A securing means is operably connected to such absorbent member at a predetermined location thereon for releasably securing such absorbent member to at least one of a person and a tube type member.

### OBJECTS OF THE INVENTION

It is, therefore, one of the primary objects of the present invention to provide a comfortable means for absorbing discharge including lochia, blood, fluid, vaginal secretions, vaginal discharge, and the like.

Another object of the present invention is to provide a device which is absorbent and which can be attached to the tubing of a catheter such that lochia, blood, fluid, vaginal secretions, vaginal discharge, and the like can be substantially absorbed.

Still another object of the present invention is to provide an apparatus which can aid in making a patient more comfortable during a time when such patient is subject to catheterization after having just given birth, being in the process of giving birth, or while menstruating.

Yet another object of the present invention is to provide a concealable absorbing device for attachment to a catheter tube for protecting bedding, flooring, clothing and the like from discharge including lochia, blood, fluid, vaginal secretions, vaginal discharge, and the like.

An additional object of the present invention is to provide an absorbing device which is attachable to a person or a catheter being used by such person such that such person is able to stand up, walk around, and change positions without fear of being embarrassed by leaking lochia, blood, fluid, vaginal secretions, vaginal discharge, and the like.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

FIG. 1 is a partial perspective view of the invention according to one embodiment of the invention in which the invention includes a slit type opening; in this illustration the slit type opening is completely closed.

FIG. 2 is a partial perspective view of the invention according to one embodiment of the invention in which the invention includes a slit type opening being flexed open for receiving such tube type member.

FIG. 3 is a partial perspective view of the invention according to one embodiment of the invention in which an aperture is centered through such absorbent member such that such tube type member will be fit through the slit type opening and housed within the apertured portion; a bottom side of the absorbent member is depicted having a fluid impervious lining thereon.

FIG. 4 is a partial perspective view of the invention according to one embodiment of the invention.

FIG. 5 is a partial perspective view of the invention according to one embodiment of the invention in which the apparatus has been attached to catheter tubing.

FIG. 6 is a partial perspective view of the invention according to one embodiment of the invention in which the apparatus has been attached to a tube type member.

FIG. 7 is a partial perspective view of the invention according to one embodiment of the invention in which a hook and loop type securing means is disposed on such absorbent member.

### BRIEF DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

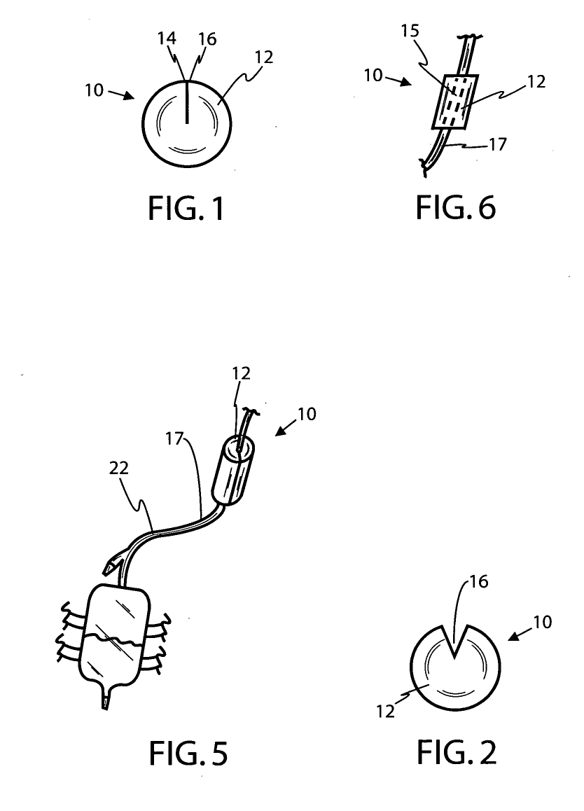

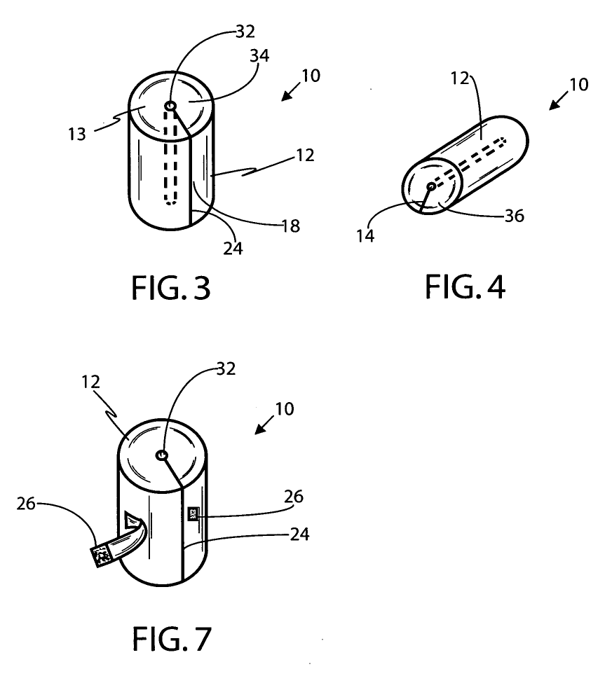

Reference is now made, more particularly, to FIGS. 1-7.

A discharge absorbing apparatus, generally designated 10, is provided.

Such discharge absorbing apparatus 10 includes an absorbent member 12 manufactured from a first predetermined material and having a first predetermined size and a first predetermined shape.

It is presently preferred that such first predetermined material is at least one of cotton, cellulose, synthetic fibers, rayon, and a combination thereof.

It is presently preferred that such absorbent member 12 is substantially cylindrical.

It is presently preferred that such absorbent member 12 is between about 0.5 inches and about 1.5 inches long.

It is further presently preferred that such absorbent member 12 is between about 1.0 and 3.0 inches in diameter. It is presently most preferred that such absorbent member 12 is about 2.0 inches in diameter.

It is presently preferred that such absorbent member 12 is flexible. It is presently preferred that such absorbent member 12 includes a plurality of layers for at least one of increasing absorbancy and reducing leakage.

It is presently preferred that such absorbent member 12 includes a fluid impervious lining 13 disposed thereon at a predetermined location. It is presently preferred that such absorbent member is odor absorbent.

A securing means 14 is operably connected to the absorbent member 12 at a predetermined location thereon for releasably securing the absorbent member 12 to at least one of a person (not shown) and a tube type member 17. It is presently preferred that such tube type member 17 is a catheter tube 22.

It is further presently preferred that such absorbent member 12 includes an opening 16 having a predetermined shape and a predetermined size extending adjacent a length of a predetermined side 18 of such absorbent member 12 and inwards a predetermined depth such that a predetermined portion 15 of a tube type member 17 having a predetermined diameter may be housed within such opening 16.

It is presently preferred that such securing means 14 is such opening 16. It is presently preferred that such depth of such opening is about 1.0 inches.

It is further presently preferred that such opening 16 is a slit type opening 24.

It is presently preferred that such absorbent member 12 includes an aperture 32 disposed therethrough from a first end 34 of such absorbent member 12 to a second opposite end 36 of such absorbent member 12 such that such tube type member 17 may be inserted into such slit type opening 24 and will substantially fit within such aperture 32.

It is presently preferred that such aperture 32 is centered within the respective ends of such absorbent member 12.

It is presently preferred that such absorbent member 12 further includes an additional securing means 26 for releasably securing such predetermined portion 15 of such tube type member 17 within at least one of such slit type opening 24 and such aperture 32. It is presently preferred that such additional securing means 26 includes at least one of an adhesive type securing means and a hook and loop type securing means.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

I claim:

1. A discharge absorbing apparatus comprising;
a. an absorbent member manufactured from a first predetermined material and having a first predetermined size and a first predetermined shape;
b. a securing means operably connected to said absorbent member at a predetermined location thereon for releasably securing said absorbent member to at least one of a person and a tube type member.

2. A discharge absorbing apparatus according to claim 1 wherein said absorbent member is flexible.

3. A discharge absorbing apparatus according to claim 1 wherein said absorbent member includes a plurality of layers for at least one of increasing absorbancy and reducing leakage.

4. A discharge absorbing apparatus according to claim 1 wherein said absorbent member includes a fluid impervious lining disposed thereon at a predetermined location.

5. A discharge absorbing apparatus according to claim 1 wherein said absorbent member is odor absorbent.

6. A discharge absorbing apparatus according to claim 1 wherein said first predetermined material is at least one of cotton, cellulose, synthetic fibers, rayon, and a combination thereof.

7. A discharge absorbing apparatus according to claim 1 wherein said absorbent member is substantially cylindrical.

8. A discharge absorbing apparatus according to claim 7 wherein said absorbent member includes an opening having a predetermined shape and a predetermined size extending adjacent a length of a predetermined side of said absorbent member and inwards a predetermined depth such that a predetermined portion of a tube type member having a predetermined diameter may be housed within such opening.

9. A discharge absorbing apparatus according to claim 8 wherein said tube type member is a catheter tube.

10. A discharge absorbing apparatus according to claim 8 wherein said opening is a slit type opening.

11. A discharge absorbing apparatus according to claim 7 wherein said absorbent member is between about 0.5 inches and 1.5 inches long.

12. A discharge absorbing apparatus according to claim 8 wherein said absorbent member is between about 1.0 and 3.0 inches in diameter.

13. A discharge absorbing apparatus according to claim 8 wherein said absorbent member is about 2.0 inches in diameter.

14. A discharge absorbing apparatus according to claim 13 wherein said depth of said opening is about 1.0 inches.

15. A discharge absorbing apparatus according to claim 8 wherein said absorbent member includes an aperture disposed therethrough from a first end of said absorbent member to a second opposite end of said absorbent member such that such tube type member may be inserted into said slit type opening and will substantially fit within such aperture.

16. A discharge absorbing apparatus according to claim 15 wherein said aperture is centered within said ends of said absorbent member.

17. A discharge absorbing apparatus according to claim 15 wherein said absorbent member further includes an additional securing means for releasably securing such predetermined portion of such tube type member within at least one of said slit type opening and said aperture.

18. A discharge absorbing apparatus according to claim 17 wherein said additional securing means includes at least one of an adhesive type securing means and a hook and loop type securing means.
